(19) United States
(12) Patent Application Publication
(10) Pub. No.: US 2012/0165949 A1
(43) Pub. Date: Jun. 28, 2012

(54) ORTHOPEDIC IMPLANT AND METHOD OF MAKING SAME

(75) Inventor: Steven L. Worthington, Angola, IN (US)

(73) Assignee: ORCHID ORTHOPEDICS SOLUTIONS, Holt, MI (US)

(21) Appl. No.: 13/336,017

(22) Filed: Dec. 23, 2011

Related U.S. Application Data
(60) Provisional application No. 61/426,715, filed on Dec. 23, 2010.

(51) Int. Cl. A61F 2/38; B21J 13/02; B21K 23/00

(52) U.S. Cl. 623/20.35; 72/357

(57) ABSTRACT

An orthopedic implant and method for forming same includes providing a forging die having a punch and a split die. The split die is movable between an opened state, where first and second die portions are spaced apart to allow for removal of a formed orthopedic implant element from within the cavity portions, and a closed state, where the first and second die portions are secured together and the cavity portions cooperate to define a cavity to form the orthopedic implant element therein. The split die is closed and a billet of biocompatible material is provided in the cavity and the punch is moved to deform the billet in the cavity to forge the orthopedic implant element in the cavity while the first and second die portions are substantially fixedly secured together when the split die is in said closed state.

### CROSS REFERENCE TO RELATED APPLICATION

The present application claims the filing benefit of U.S. provisional application Ser. No. 61/426,715, filed Dec. 23, 2010, which is hereby incorporated herein by reference in its entirety.

### FIELD OF THE INVENTION

The present invention relates generally to orthopedic implants and methods for making orthopedic implants and, more particularly, to forged orthopedic implants forged from a biocompatible metallic material.

### BACKGROUND OF THE INVENTION

Orthopedic implants are typically formed from a metallic material that is biocompatible, such as, for example, cobalt-chrome, titanium, stainless steel or zirconium and/or alloys thereof. Often, such metallic materials are difficult to form to the desired shape with known forming methods, including casting and forging of these metallic materials. However, cast implants tend to have less strength than forged implants of similar materials and size and shape. While the desire to forge complex-shaped implants using such biocompatible materials has existed, it has to date been difficult to achieve the desired component characteristics and strength via such forging processes. Also, such forging processes typically result in a substantial amount of wasted material, with the finished component often being in the range of only about 30-40 percent of the material of the original billet that undergoes the forging process (resulting in a material utilization of less than around 40 percent for forging orthopedic implants).

Prior known forging processes (such as, for example, an open die forging process of the type described in U.S. Pat. No. 7,168,283, which is hereby incorporated herein by reference in its entirety) use two separate or open die parts whereby the forging stock or material or billet that is to be forged is placed in the partial cavity of one of the die parts (after initial heating of the billet to a desired or appropriate temperature) and the die parts are rapidly pressed together to form the part. During such known punching and/or pressing processes, a substantial amount of material rapidly flows outward between the open spaced apart die parts as the die parts are punched or pressed together. The rapid and excessive amount of material flow outward from between the die parts limits the amount of pressure that can be applied during forging to the component being formed and, due to the extreme temperatures achieved during the rapid flowing of the material (due to the rapid punching or pressing of the dies together during known forging processes), the excess material is typically thermally damaged and cannot be salvaged for future use. The forged component that is forged via such known open die processes thus has a significant amount of flash or excess material about its periphery that has to be machined or ground off to form the finished component. Also, due to the higher temperatures or extreme temperatures that the periphery of the component achieves at the flash regions (where the flow of the material is the greatest), the finished component (after the flash has been machined or ground off) may have flow lines or witness lines along the flash regions that reflect the thermally damaged material.

Therefore, there is a need in the art to manufacture orthopedic implants using a forging technique that provides enhanced product characteristics and enhanced material utilization.

### SUMMARY OF THE INVENTION

The present invention provides an orthopedic implant element (such as, for example, a femoral knee component or femoral component or the like formed from a biocompatible metallic material) that is forged in a split die and multi-axis forging process or system, whereby the forging process achieves a material utilization of at least about 60 percent (such as about 80 percent or more material utilization) and provides a finished product that has enhanced material characteristics. The split die, multi-axis forging system allows for high pressure forging (such as achieving pressure greater than about 200,000 psi, such as at least about 250,000 psi or thereabouts) of the orthopedic implant, which results in a finished forged orthopedic implant having reduced or finer grain size and substantially uniform or homogeneous material composition and enhanced surface finish.

The orthopedic implant may comprise any suitable biocompatible metallic material, such as zirconium or a zirconium alloy or titanium, a titanium alloy, stainless steel, a stainless steel alloy, cobalt-chrome or a cobalt-chrome alloy or the like. The orthopedic implant is formed via a high pressure forming or forging process and thus has enhanced or finer grain size. For example, the finished forged orthopedic implant (forged via the forging method or system of the present invention) may have a grain size of about G16.0 or finer substantially throughout the orthopedic implant element.

These and other objects, advantages, purposes and features of the present invention will become apparent upon review of the following specification in conjunction with the drawings.

### BRIEF DESCRIPTION OF THE DRAWINGS

FIG. 1 is a perspective view of a forged orthopedic implant or femoral knee component in accordance with the present invention;

FIG. 2 is a plan view of the forged femoral knee component of FIG. 1;

FIG. 3 is a sectional view of the femoral knee component of FIG. 2;

FIG. 4 is a side elevation of the forging system or apparatus for forging the orthopedic implant in accordance with the present invention;

FIG. 5 is a plan view of one of the die portions of the forging apparatus;

FIG. 6 is a side elevation and partial sectional view of the forging apparatus of the present invention;

FIG. 7 is a schematic showing the die cavity and punch of the forging process, with a unformed generally cylindrical billet disposed in the cavity;

FIGS. 8-11 are schematics of the forging process, showing the punch progressively moved downward to deform the billet within the cavity;

FIG. 12 is another schematic of the forging process, showing the punch in its fully extended or stroked position to form the orthopedic implant in the cavity in accordance with the present invention;

FIGS. 13 and 14 are photographs of a sample forged femoral knee component forged via the forging process of the present invention, shown with lines drawn along the forged part where the forged part was cut for metallurgical analysis of the forged femoral knee component;

FIG. 15 is a photograph of the cut femoral knee component to show one of the sections of the femoral knee component, with reference to the regions of the cut component that were analyzed and shown in photomicrographs in FIGS. 17 and 18;

FIG. 16 is a photograph of another cut portion of the femoral knee component, with reference to the regions of the cut component that were analyzed and shown in photomicrographs in FIGS. 19-21;

FIGS. 17-21 are photomicrographs of portions of the cut sections of the cut sections of the femoral knee component of FIGS. 15 and 16, showing the grain structure at the respective regions noted in FIGS. 15 and 16;

FIG. 22 is a photomicrograph of the grain structure of a billet of the type used to forge the femoral knee component of FIGS. 13 and 14;

FIG. 23 is a photomicrograph of the grain structure of a femoral knee component forged by a known open die forging process;

FIG. 24 is a photomicrograph of the grain structure of a femoral knee component forged via the split die high pressure forging process in accordance with the present invention; and

FIG. 25 is a schematic showing the grain flow pattern of a femoral knee component forged via the split die high pressure forging process in accordance with the present invention.

### DESCRIPTION OF THE PREFERRED EMBODIMENTS

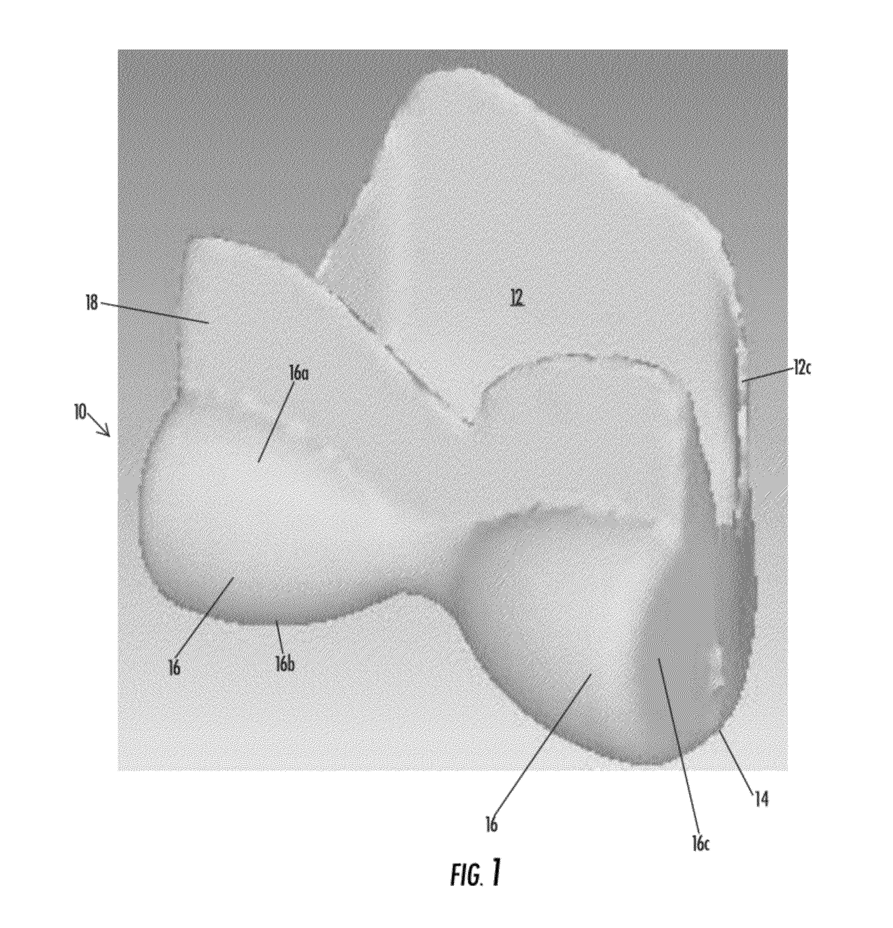

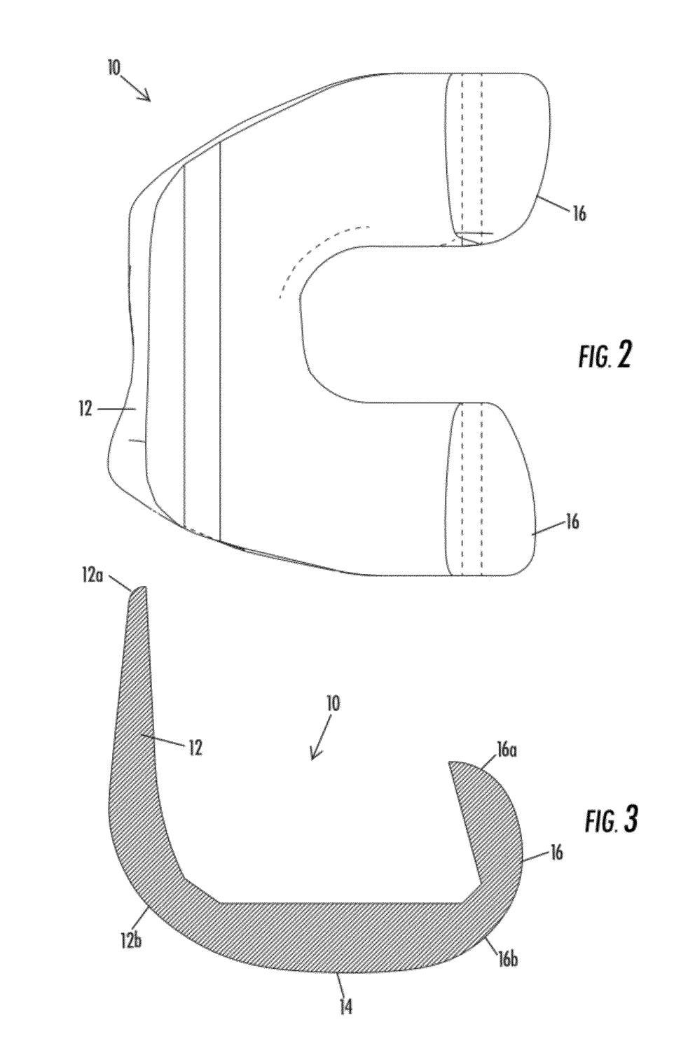

Referring now to the drawings and the illustrative embodiments depicted therein, an orthopedic implant 10 (FIGS. 1-3) is forged in accordance with the present invention via a forging system or method that provides enhanced material utilization (and thus less wasted material) and enhanced finished product material characteristics, including greater material density and finer grain size, along with enhanced surface finish and enhanced or improved tribological interaction for the finished component, as discussed below. In the illustrated embodiment, the orthopedic implant comprises a femoral knee component or femoral component, but aspects of the forging process of the present invention are equally suitable for forming or forging other orthopedic implants, while remaining within the spirit and scope of the present invention. The forged orthopedic implant of the present invention comprises any suitable biocompatible material, such as a biocompatible metallic material, such as, for example, cobalt-chrome and/or alloys thereof, titanium and/or alloys thereof, zirconium and/or alloys thereof (such as, for example, zirconium 2.5 niobium or Zr 2.5 Nb or the like), and/or stainless steel and/or alloys thereof. Such biocompatible metallic materials, and particularly cobalt-chrome, can have substantially high flow stresses (the resistive force that has to be overcome to deform the part). For example, a preferred cobalt-chrome metal material for a femoral knee component may have a flow stress of, for example, about 40 ksi when heated to a temperature of about 2000 to 2200 degrees F. (with cobalt-chrome having a substantially high flow stress or resistance to material flow and with stainless steel typically having a lower flow stress than cobalt-chrome and titanium having a lower flow stress than stainless steel and zirconium having a lower flow stress than titanium). Another preferred material is zirconium and/or alloys thereof, which testing has shown to provide an enhanced component via the forging method of the present invention.

As shown in FIGS. 1-3, the forged femoral knee component 10 comprises an anterior flange 12, a lower articulating surface 14 and a pair of condyles 16. The outer surface of the forged femoral knee component comprises multi-radius or complex curvatures at opposite sides of the forged part. For example, the forged femoral knee component 10 (shown in FIG. 1 as it is removed from the forging die of the present invention and with its flash material 18 (the material that flows out of the forging die cavity during the forging process) and before grinding or machining of the part to its final finished form), has an upper curved surface 16a at the upper region of the condyles 16 and a lower curved surface 16b at the lower region of the condyles 16 and side surfaces 16c at the sides of the condyles, and also has an upper curved surface 12a at the upper region of the anterior flange 12 and a lower curved surface 12b at the lower region of the anterior flange 12 and side surfaces 12c at the sides of the anterior flange 12. The complex shape of such a component (with curved surfaces on all sides) makes it difficult to forge such a component via conventional split die forging techniques without a significant amount of flash material and thus a significant amount of wasted material and a significant amount of post-forge processing (such as grinding and polishing) of the part to form the finished product.

Because of the complex shape of the part, prior known forging processes (such as discussed in the Background section above) may use two separate or open die parts to forge the stock or material or billet to the desired form and forge or form the part by rapidly pressing together the die parts. Such known or typical forging processes can produce a forged component that may be stronger than an equivalent cast or machined part, and the grain structure of a typical forged part may be improved as compared to cast or machined parts. However, such improvements are limited in known forging processes due to the lower pressures achieved at or on the part being formed and the rapid and substantial flow of excess material between the open dies as they are pressed together (because of the open dies and large gap between the dies for the flowing of the excess material or flash), with the pressure on the material during the forging process being unable to reach the levels that can now be achieved by the split die forging system or process of the present invention, as discussed in detail below.

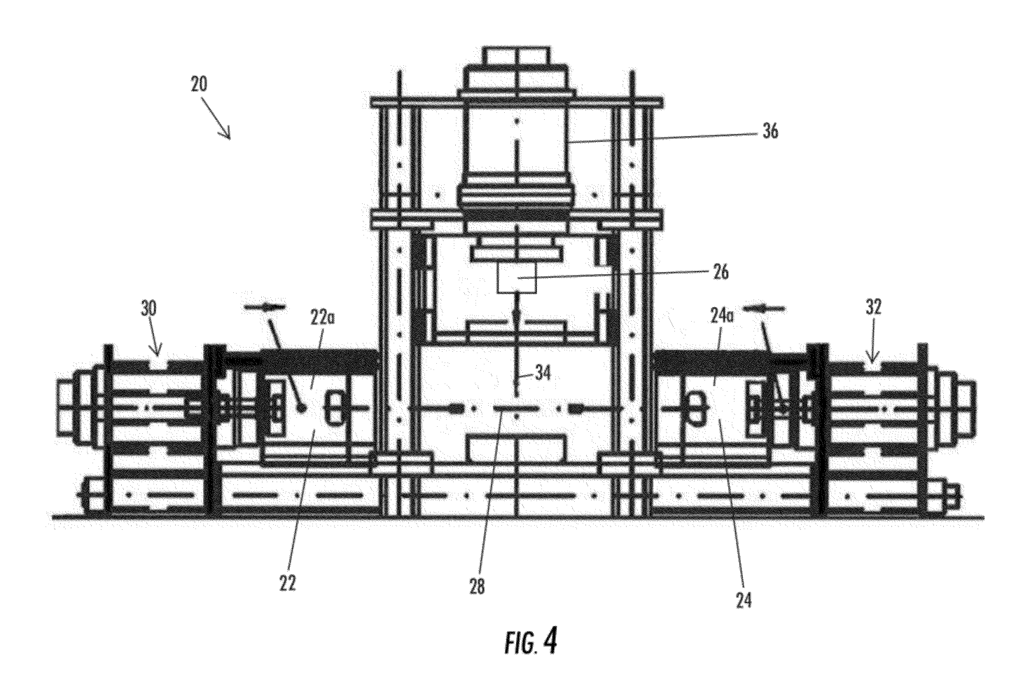

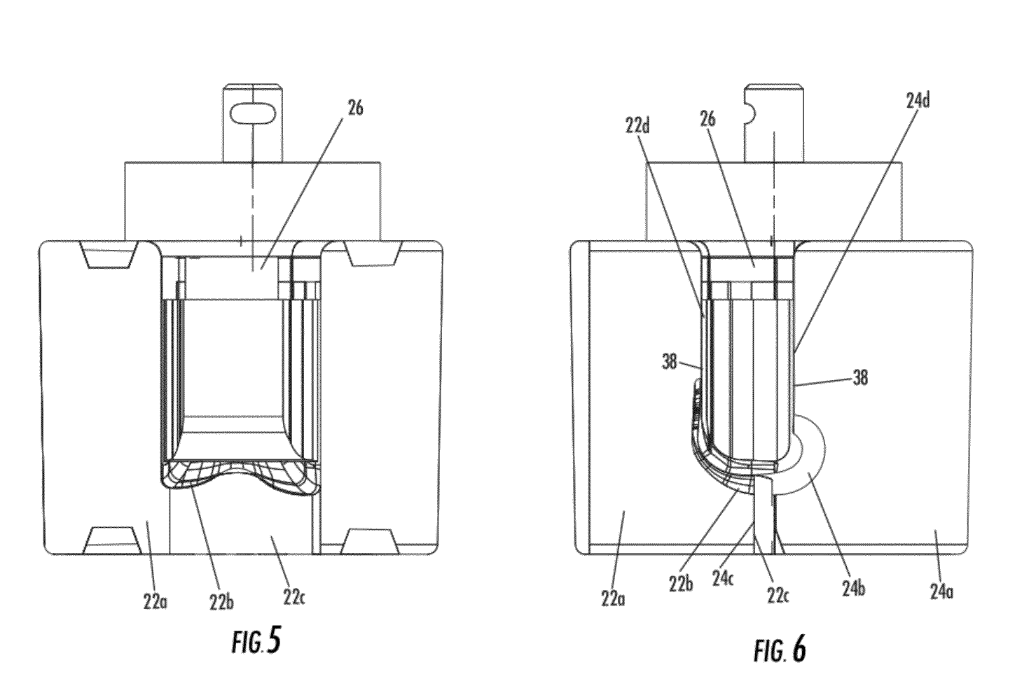

Referring now to FIGS. 4-6, a multi-axis split die forging system or apparatus 20 includes a first die portion 22, a second die portion 24 and a punch 26. The first and second die portions 22, 24 include substantially rigid platens or bodies 22a, 24a with a recess or cavity portion 22b, 24b formed therein at opposing surfaces 22c, 24c of the bodies or platens 22a, 24a (FIG. 6). In the illustrated embodiment, each of the first and second die portions 22, 24 is movable along a die opening-closing axis 28 (shown as a generally horizontal axis in FIG. 4) via respective hydraulic rams 30, 32. The punch 26 is movable along a punch axis 34 (shown as a generally vertical axis in FIG. 4) that is generally transverse to the die opening-closing axis 28 and is movable via a punching apparatus or device or actuator 36. Each of the die portions 22, 24 comprises its respective cavity portion 22b, 24b for forming a portion (such as opposite side portions or shapes) of the finished orthopedic implant or femoral knee component during the forging process. For example, for forging a femoral knee component, the first cavity portion 22b may define the condyles of the femoral knee component and the second cavity portion 24b may define the anterior flange of the femoral knee component, with each of the cavity portions defining a respective portion of the articulating lower surface of the femoral knee component. Because each die portion and respective cavity portion defines or forms curved surfaces at the upper and lower portions of the formed component, it is desirable that the die comprises a split die to facilitate forging of the component and removal of the forged component after it is formed within the closed die. The die portions 22, 24 include recesses 22d, 24d established therein for receiving a portion of the punch 26 as the punch is moved into the die cavity to form the component, as discussed below.

The actuators or hydraulic rams 30, 32 that move the die portions 22, 24 towards one another and retain the die portions 22, 24 together during the forging process comprise high pressure/high force output hydraulic rams that use non-compressible or substantially non-compressible pressurized fluid, such as known hydraulic fluids and the like. The actuators are operable to exert substantial forces (such as a force greater than about 1.5 times the force exerted by the punch, such as, for example, a force greater than about 750 U.S. tons or thereabouts along the opening-closing axis 28 when the punch 26 exerts a force of greater than about 500,000 pounds along the punch axis 34) to move the die portions toward one another and against one another and to hold the die portions together (with the non-cavity or non-recessed opposing portions or surfaces 22c, 24c of the platens pressed tightly together) during the forging process. Because the pressures in the die cavity during the forging processes may exceed about 200,000 psi and may exceed about 250,000 psi (as discussed below), the hydraulic rams operate to maintain the die portions pressed tightly together to that little or no material escapes the die cavity at the interface between the die portions and respective platens during the forging process.

Optionally, and desirably, the forging system 20 includes a control device and sensors that determine the movement and location of the die portions (such as via detecting the location of the die portions or detecting a level of extension/retraction of the respective hydraulic rams or the like). The control is responsive to outputs of the position or extension/retraction sensors and functions to synchronize the extension of the hydraulic rams to make sure the die portions are positioned properly relative to one another with their center line or interface surfaces at appropriate location so that, when the die portions are clamped or pressed together to close the die, the opening to the die for the punch is at precisely the appropriate location relative to the punching axis to receive the punch therein and therealong when the punch is moved along the punching axis toward and into engagement with the billet disposed within the cavity of the closed split die.

Optionally, the control may operate to actuate the hydraulic rams or actuators to move the respective die portions to engage one another at the precise location where the opening to the die cavity is aligned with the punch, and optionally, a positive stop element may be disposed at the forging apparatus to limit movement of either or both of the die portions beyond the precise location where the opening to the die cavity is aligned with the punch. Optionally, the opposing surfaces of the die portions may include one or more male-female guide elements that engage one another as the die portions are urged together to ensure that proper alignment is achieved and maintained during the die closing process. For example, one of the die portions may include one or more conical-shaped protrusions that are received in similar or correspondingly-shaped recesses to align or maintain alignment of the die portions as they are pressed together.

The dies or die portions are moved together and are locked or secured or retained together by the hydraulic rams or presses, which provide a hydraulic clamp to secure the die portions together during the forging process. Although shown and described as hydraulic rams that move the die portions together and hold the die portions together during the forging process, it is envisioned that other means may be implemented to move and secure the die portions, such as mechanical means or electro-mechanical means or the like. For example, a linear actuator or rack and pinion type mechanical or electro-mechanical means may be implemented to move the die portions, and optionally, a mechanical lock or latch may be used to retain the die portions together during the forging process, while remaining within the spirit and scope of the present invention. However, because of the significant forces and pressures achieved within the die cavity during the forging process of the present invention, the means for moving and securing the die portions or sections together are preferably hydraulic means, such as hydraulic rams or actuators or cylinders, which limit or substantially preclude any elastic deformation or movement of the die portions during the forging process.

When the die portions are pressed together and the die cavity (with the billet disposed therein) is closed, the punch is moved into the die cavity through the opening established between the die portions. The punch is then driven or moved further into the cavity and into engagement with the billet to deform the billet and form or forge the component. The punch recesses 22d, 24d in the die portions 22, 24 cooperate to form an opening or passageway to the die cavity that is configured to receive the punch therein. When the punch is disposed in the opening or passageway formed by recesses 22d, 24d, a gap or flash gutter 38 is established between the walls or side surfaces of the punch 26 and the surfaces of the die portions at the recesses 22d, 24d, with the gap or gaps 38 allowing for flow of excess material out of the die cavity during the process of forming or forging the orthopedic implant. The gaps 38 provide narrow escape passageways for the excess material and function to limit escape of the material until a desired or appropriate pressure is achieved in the die cavity during the forging process.

The forging system of the present invention thus provides a high pressure multi-axis precision forming or forging process, where the die portions do not move relative to one another during the forging process, even though the forging process may achieve pressures in the die cavity of greater than about 200,000 psi, optionally and desirably greater than about 250,000 psi, and optionally and desirably greater than about 300,000 psi (depending in part on the material properties of the material selected for the forged component). Testing and computer modeling has found that, in order to achieve such high internal cavity pressures, the punch may operate to exert a force on the billet/formed part in the range of at least about 400,000 lbs, more preferably at least about 500,000 lbs and optionally at least about 600,000 lbs or more (depending in part on the material selected and the complexity of the shape of the part to be formed), while the hydraulic rams may operate to exert a clamping force at the die portions in the range of about 600 U.S. tons, more preferably at least about 750 U.S. tons and optionally at least about 900 U.S. tons (with the force exerted by the punch being less than the force required to secure the die portions together, such as less than about ⅓ to ½ (or more or less) of the force required to secure the die portions together). Such high forces are used to overcome the flow stress and subsequent cavity pressure as the biocompatible material is being shaped and forged.

For example, computer modeling of forging a titanium femoral knee component using the forging process of the present invention has shown that, in order to achieve a desired cavity pressure of about 250,000 psi, the punch may exert a force of about 500,000 lbs (such as, for example, about 523,000 lbs). For titanium, the flow stress of the material is about 10,000 psi when the material is heated to a temperature in the range of about 1300 degrees F. to about 2300 degrees F., such as, for example, about 1700 degrees F. (which is a suitable initial heating temperature for heating the billet before the forging process begins). The cavity pressure in this example is thus about 25 times that of the flow stress for the material being formed. Thus, the complexity of the part being formed (for example, a titanium femoral knee component) may require a force or pressure equal to the flow stress times 25 or thereabouts. Thus, for materials with greater flow stresses at a given working temperature (for example, 1700 degrees F. or thereabouts), a greater pressure (such as greater than 250,000 psi) may be preferred (and may be achieved via a greater force exerted by the punch and/or smaller flash gutters or gap dimensions at the punch opening of the die cavity, such as discussed below) to overcome the flow stress of the material and to properly form the material to the desired shape while achieving the desired enhanced material properties (for example, finer grain size, reduced imperfections, homogeneous material composition and the like).

Due to the high pressures achieved in the die cavity during the forging process, and as the metal forging stock is shaped to the final form during the forging process (as the forces and pressures overcome the flow stress of the material being formed), the internal grain structure of the material deforms to follow the general shape of the part, with the die structure limiting escape of flash material to enhance the pressures in the die cavity. As a result of the high pressure forging process of the present invention, the grain structure of the forged part is substantially continuous or uniform throughout the forged part, giving rise to a final product with improved strength characteristics (particularly as compared to conventional forged parts or cast parts or machined parts), as discussed below.

Because of the high pressures and forces achieved during the forging process of the present invention and because of the types of materials being forged into the orthopedic implant, the die materials are selected to be materials that exhibit a balance of strength, toughness and wear resistance at elevated forming temperatures to assure financial viability for the forging system. For example, the die portions may comprise a suitable high strength steel, such as H-19 or H-13 steel or the like (with or without coatings to enhance wear resistance given the cavity pressures and temperatures experienced during forging depending on material being formed). The punch material may also comprise any suitable high strength material, preferably a material that exhibits high compressive strength and wear resistance, such as, for example, an S-7, D-2 and/or M-2 steel or the like. Like the forming die, the punch may be produced with or without coatings to enhance high temperature hardness given the cavity pressures and temperatures that may be experienced by the punch during the forging process (depending on the particular application and the material being formed into the particular orthopedic implant element). Optionally, and desirably, the lower or engaging end of the punch may have a roughened texture or non-uniform surface pattern or the like established thereat to create a non-uniform or roughened or patterned inner surface of the forged femoral component, which may function to enhance bone growth at and onto the femoral component after the component is implanted at the end of the patient's femur during the knee replacement surgery.

During the forging process, and after the forging stock or heated billet (such as a generally cylindrical billet cut from rod stock and heated to a desired temperature, such as at least about 1700 degrees F. or more or less depending on the selected material and desired or appropriate flow stress and/or the like) is placed in the die cavity and the die portions are closed and secured together, the punch 26 is received in an opening at and between the closed or secured together die portions and is movable along the punching axis 34 via the punching apparatus or device 36, whereby movement of the punch along the punching axis causes the punch to engage the billet disposed within the cavity of the closed split die to deform the billet material into the final forged orthopedic implant or femoral knee component.

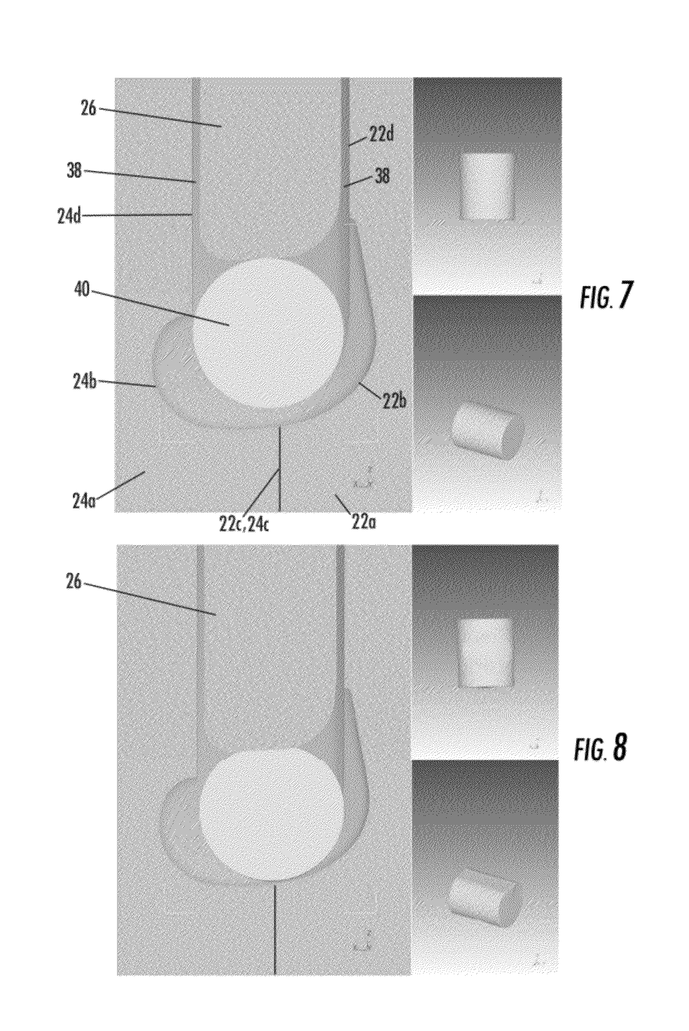

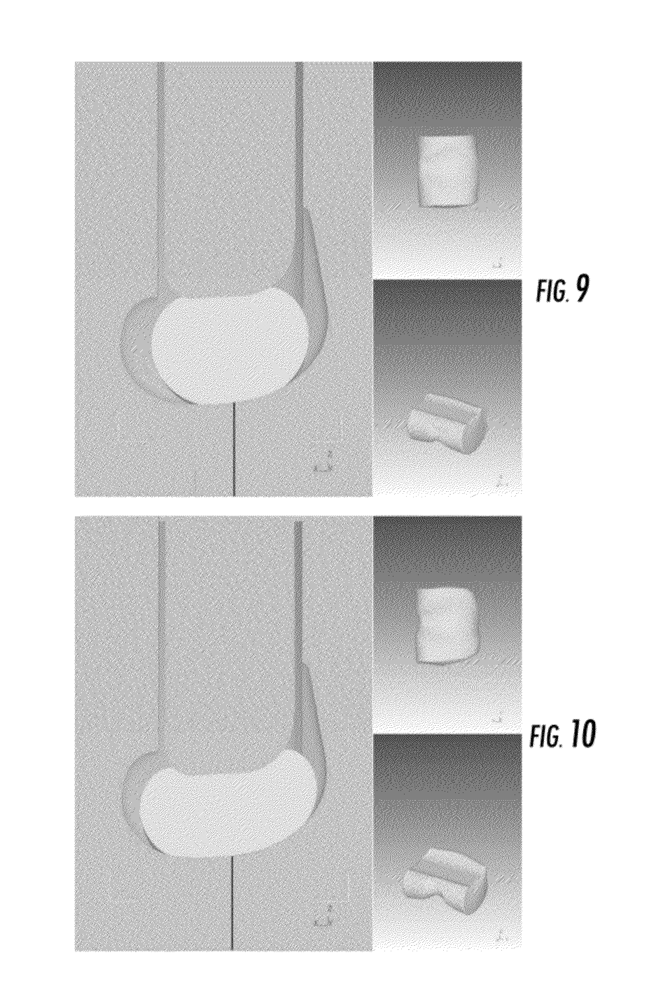

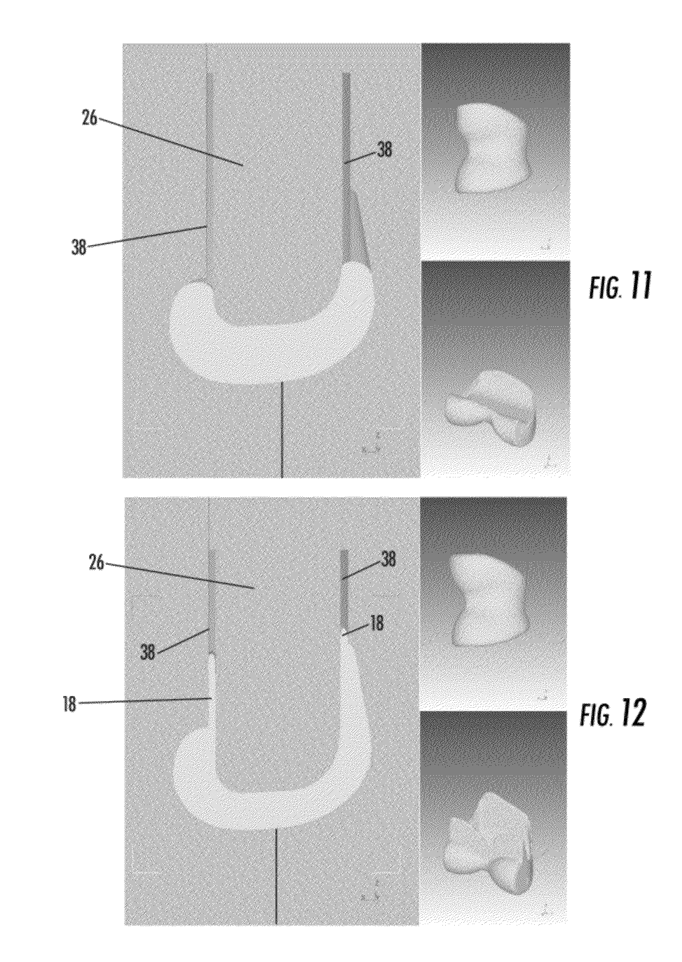

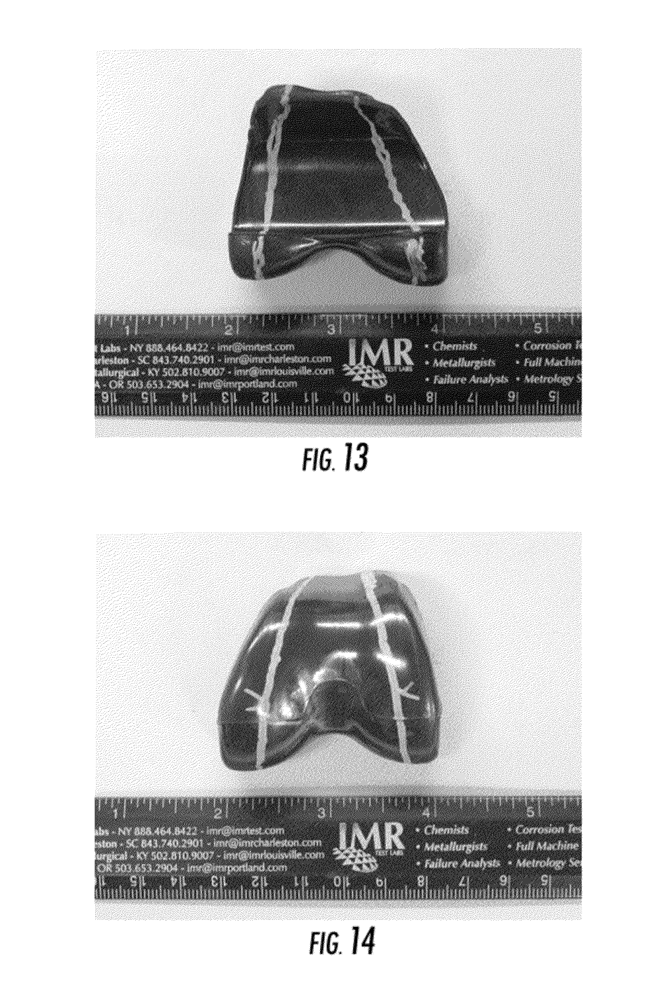

Referring now to FIGS. 7-12, the process of forging a generally cylindrical billet or forging stock 40 into the finished forged orthopedic implant is shown. As shown in FIG. 7, the cylindrical billet or forging stock is disposed or received in the die cavity (it is envisioned that the billet may be disposed in one of the cavity portions before the die is closed or may be dropped into or disposed in the cavity through the punch hole or passageway after the die is closed) and the punch is moved along the punching axis to engage the billet disposed in the die cavity. As the punch is moved further along the punching axis (such as shown in FIG. 8), the billet begins to deform, and deforms further as the punch continues to move along the punching axis (FIG. 9). Further movement of the punch causes the billet to begin to flatten and the material to flow toward and into the condyle portions and anterior flange portions of the die cavity (FIGS. 10 and 11). When the punch has reached its finished stroke position (i.e., when the punch actuator is fully extended as shown in FIG. 12), the orthopedic implant or femoral knee component is formed within the die cavity, with excess material or flash 18 flowing along the gaps or flash regions of the forging system and between the die portions and the sides of the punch.

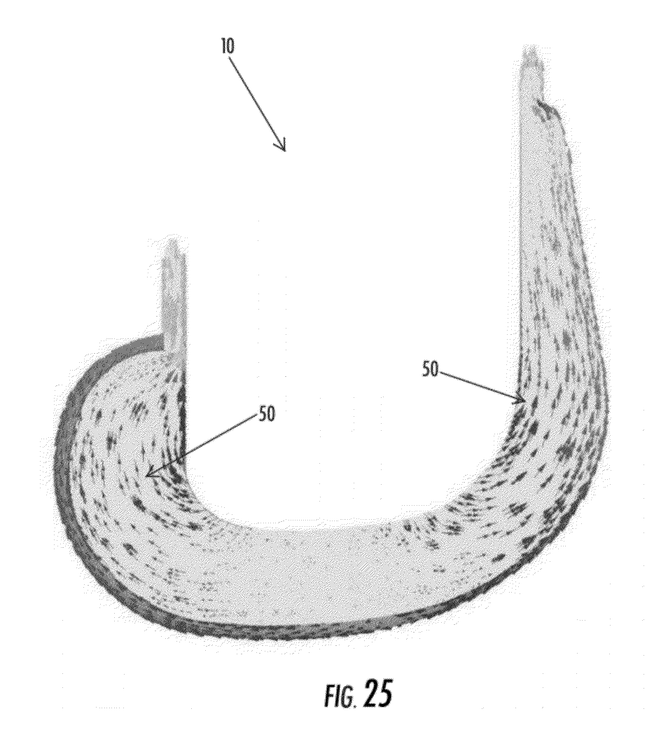

As can be seen in FIG. 25, the flow direction or flow pattern 50 of the material in the forging cavity during the forging process is generally along and generally parallel to the cavity walls or cavity profile as the material is pressed and formed within the cavity and as the material flows around the punch and towards the flash gaps (and in a generally U-shaped pattern for the forged femoral knee component 10). Thus, the present invention provides enhanced flow patterns as compared to the prior art open die forging processes, which may have the flow direction of the material generally towards the separate die portions and thus generally perpendicular to the die profiles or walls (and thus generally transverse to the surface of the forged part). The enhanced flow pattern of parts forged in accordance with the present invention (having the flow direction or pattern generally parallel to the cavity profile and generally parallel to and along the surface of the forged part) provides a general alignment of the metal microstructure with the surface of the part being forged, which may provide enhanced strength, toughness, fatigue resistance and higher life cycles for the forged parts as compared to conventionally forged parts that are forged via conventional split die forging processes.

As can be seen in FIGS. 6 and 12, the die portions 22, 24 define respective portions 22b, 24b of the die cavity for forming the finished part, and also have recesses 22d, 24d at their upper regions and above the cavity portions for receiving the punch 26 and receiving and allowing for flow of excess material or flash out of the cavity during the forming process. The recesses 22d, 24d along and between the die portions and punch provide narrow gaps between the surface of the die portions above or adjacent to the part-forming cavity and the respective sides of the punch 26 as the punch is moved along the punching axis to form the orthopedic implant and the cavity of the split die. The size or gap dimension of the gap or flash gutter between the respective die portion and the side of the punch is preferably small enough (such as on the order of about 0.040 inches to about 0.060 inches) so that the material will not flow into the gaps or will be limited in flow into the gaps until or while the pressure in the die cavity reaches a desired level, such as, for example, a pressure of at least about 200,000 psi or 250,000 psi or thereabouts, during the forging process. The cavity pressures achieved during forging and the gap or flash gutter dimensions may vary depending on the shape and desired characteristics of the part being formed and depending on the material used to form the part.

Because the flash gutters or gaps (between the die portions and the punch when the punch is moved toward and into the die cavity) provide the only escape of material during the forging process (when the die portions are tightly clamped together so that virtually no material escapes between the interface surfaces 22c, 24c of the die portions), the size or gap dimension of the gaps comprises or provides a selectable or adjustable process parameter that effectively dictates the cavity pressure within the die cavity during the forging process. Thus, a narrower or smaller gap (such as a gap size of about 0.020 inches to about 0.040 inches or thereabouts) would result in a greater or higher pressure during the forging process, which results in a thinner flash material and further enhanced or smoother finish of the forged part (as compared to a larger gap size of greater than about 0.040 inches, such as a gap size of between about 0.040 inches and about 0.060 inches or about 0.080 inches or thereabouts, which would result in a reduced pressure, but still substantially greater pressure than those achieved via conventional forging processes, and thicker flash material at the forged part).

Such higher pressure forgings provide for a more uniform density component with reduced grain size and enhanced surface finish over lower pressure forging processes known in the art (and such higher pressures cannot be achieved via conventional open die forging processes). Optionally, the gap dimension may be increased or otherwise selected to the desired dimension to provide the desired finished forged product characteristics. Optionally, it is envisioned that the gap size may be selected or adjusted, such as via grinding the punch or die platens to increase the gap and thus increase the flash thickness and decrease the cavity pressure achieved during the forging process.

The gap dimension thus may be a calculatable parameter that can be determined and set to provide a forged part having the desired grain size and surface finish. It is envisioned that an algorithm may provide a calculation (for a given part shape and material used) that determines or provides (responsive to an input of the desired grain size and surface finish and material or type of material or material properties) an appropriate gap dimension for a forging system that will forge the part and achieve the desired and input/selected part characteristics. In other words, for a desired grain size and surface finish of a forged part, the flash gaps or gutters may be readily determined and designed to achieve the desired results. For example, if it is desired to have a smaller grain size and smoother surface finish, the gap size can be selected or reduced an appropriate amount to provide the desired results. Thus, the present invention provides a configurable and adjustable or customizable high pressure forging system that allows a customer to input or provide the desired part characteristics, whereby the system is readily configured or designed or customized to forge parts that achieve the desired part characteristics.

Because of the high pressure achieved during forging with the forging system of the present invention, the forged orthopedic implant has enhanced and desirable material properties and characteristics. For example, the forged orthopedic implant may have a finer grain size and more uniform metallurgical properties throughout the forged part as compared to conventional forged or cast components. Also, the orthopedic implant forged via the forging system of the present invention has an enhanced surface finish, which provides substantially reduced surface imperfections or irregularities. The forged orthopedic implant as forged in accordance with the present invention also provides a more uniform microstructure, all of which provide for enhanced strength of the forged component and enhanced wear or enhanced tribological interaction of the finished product.

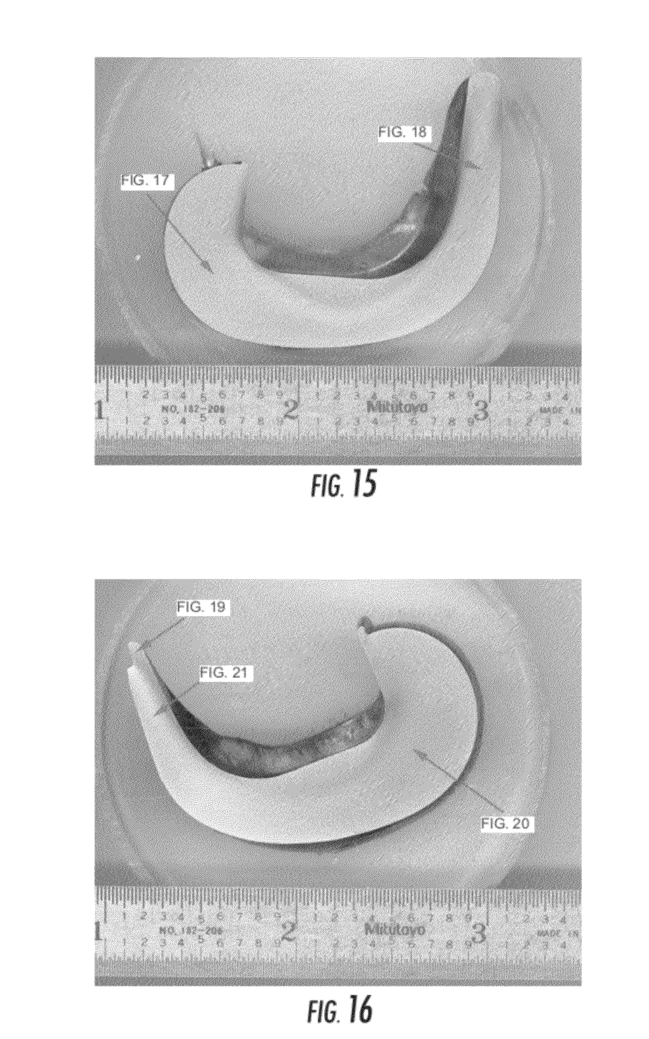

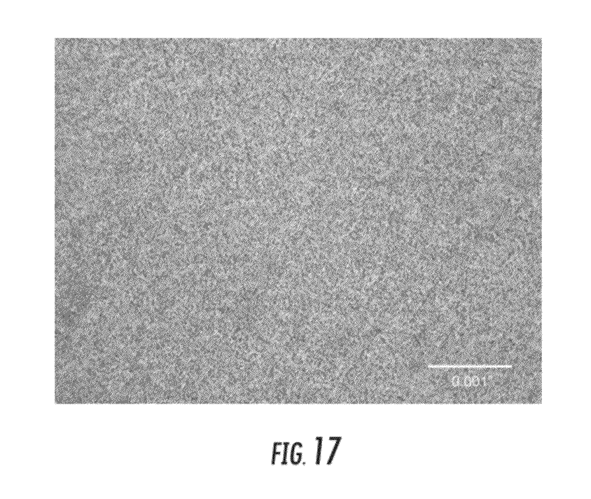

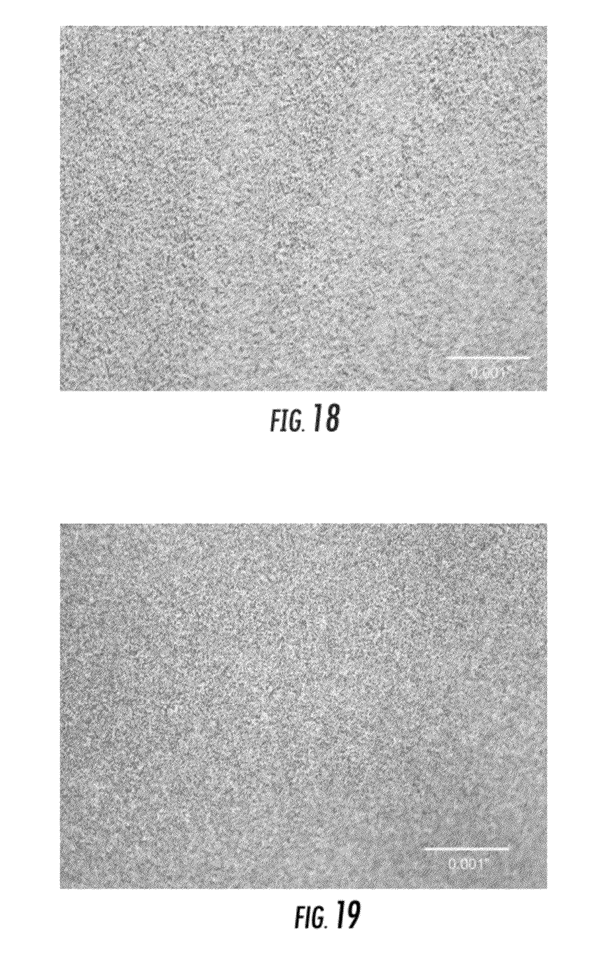

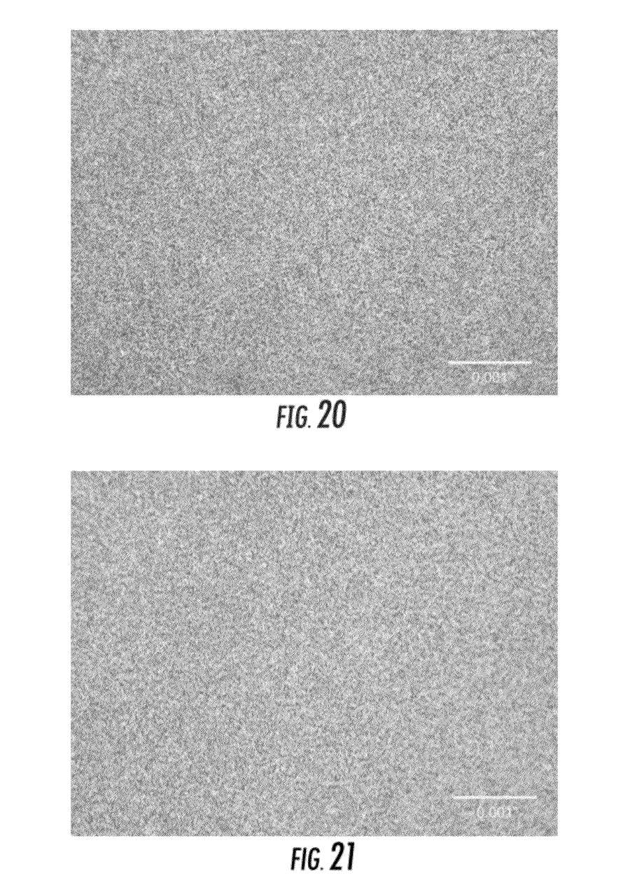

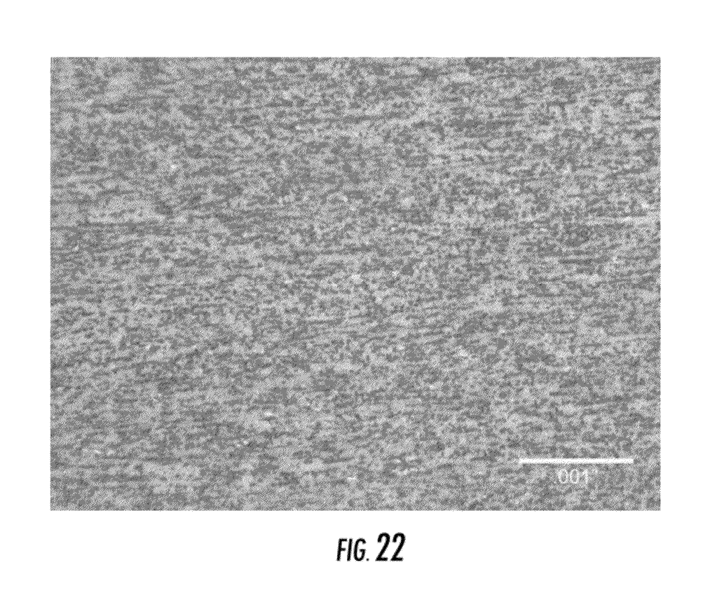

Testing of sample components forged via the forging system and method/process of the present invention has shown that the forged part has improved or enhanced material characteristics. For example, and with reference to FIGS. 13-21, a sample part is shown (FIGS. 13 and 14) that was cut along two cut lines A, B, and the material of the cut part was analyzed at the locations shown in FIGS. 15 and 16 (with the representative photomicrographs showing typical green structure of the samples at the respective locations shown in FIGS. 17-21). The sample part comprised a zirconium-2.5 nobium material (Zr 2.5 Nb). The cut samples were examined at 800× magnification in accordance with ASTM E112-96 (2004). The results of the analyses indicated that the grain size at each location checked was about G16.0 or finer (the G value or size characterization of grains increases as the size of the grains decreases, so, for example, a grain size of G16.0 is substantially finer or smaller than a grain size of G14.0, and thus because the grains analyzed were greater than about a G16.0 grain size or rating or value, the measured grains were smaller or finer than grains with a G16.0 grain size). This is a substantial reduction in grain size as compared to similar components formed via known or conventional open die forging processes. For example, the grain size or specification for a typical part formed via known open die forging processes is about G10 to about G12.0, which is a grain size that is about 70 percent larger or greater than the measured G16.0 grain size of the sample parts formed in accordance with the present invention (a G16.0 grain size is substantially finer than a G12.0 grain size). As can be seen in FIGS. 17-21, the grain structure and size are substantially uniform throughout the forged part, such that the forged orthopedic implant of the present invention has a substantially uniform part composition.

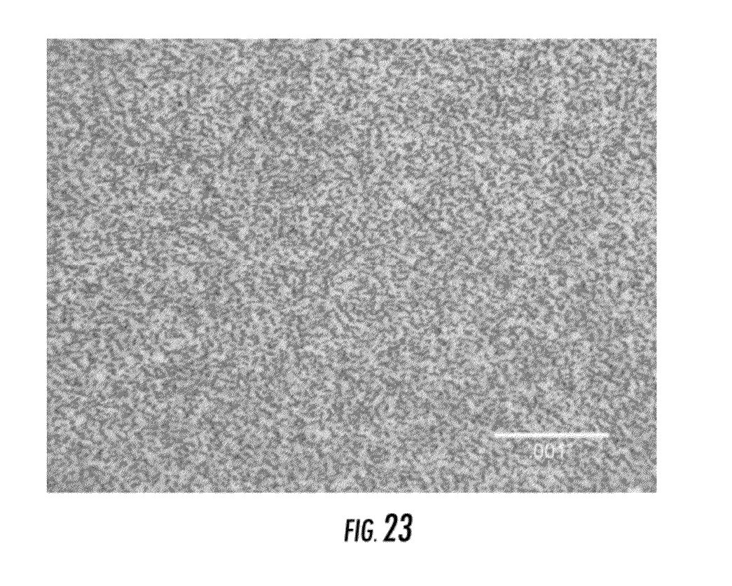

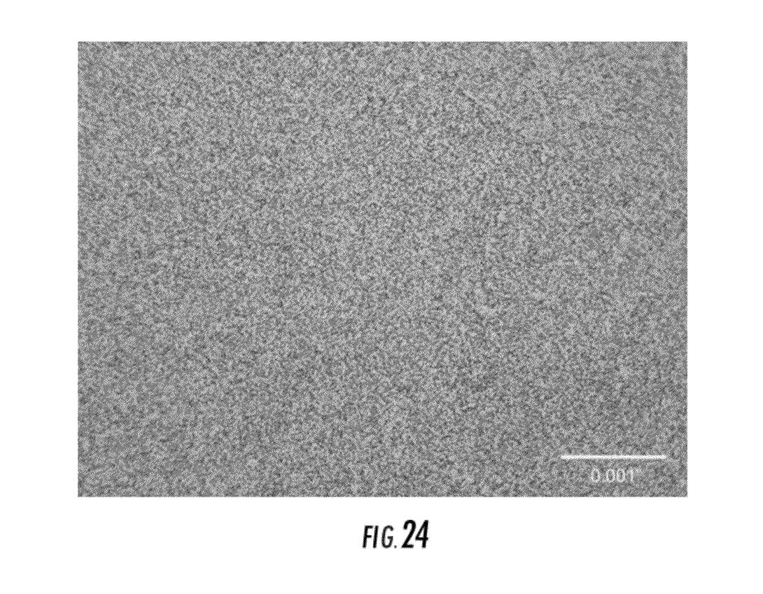

The forging process of the present invention thus provides enhanced more homogeneous microstructure of the forged component. For example, and with reference to FIGS. 22-24 (showing a comparison of microstructures of bar/billet stock, a product formed via open-die forging and a product formed via the high pressure forging system of the present invention), forging stock of a Zirconium 2.5 Niobium bar has a larger grain size (as shown in the photomicrograph in FIG. 22 of a bar/billet stock of Zr 2.5 Nb alloy) than the material has after such a forging stock is forged into an orthopedic implant via known or conventional forging processes (as shown in the photomicrograph in FIG. 23 of a product formed via open die forging). However, when such a forging stock is forged into an orthopedic implant via the multi-axis high pressure forging system of the present invention, the forging system may deform the material and work the material so that the forged product has a grain size of about G16.0 (or a substantially smaller grain size), such as shown in the photomicrograph in FIG. 24 of a product formed via the forging system of the present invention.

Based on a review of these samples, it is apparent, and such as reflected in the photomicrographs of FIGS. 22-24, that there is an increased level in material recrystallization achieved by the high pressure forging process of the present invention. These differences can be seen by comparison of the open-die forging process microstructures and the high pressure forging process microstructures. The increased level of grain refinement associated with the high pressure forging process of the present invention is not only a characteristic result of the process, but such grain refinement results in a more homogenous structure and improved tribological interaction between the forged part (such as a femoral knee component for a total knee replacement) and the surface that the forged part engages (such as the tibial plate or articulating surface at the tibial plate of the total knee replacement).

The increased level of grain refinement results from the ability to generate higher cavity pressures via the forging process of the present invention as compared to known split die forging processes used to form orthopedic implants out of biocompatible metals. Likewise, the high pressure forging process of the present invention provides enhanced surface finish to the forged component. The surface finish relating to the parts formed with the high pressure forging process is a consequence of the ability to control the cavity pressure via the flash gutter design of the forging system of the present invention. Maintaining a thinner gap or gutter results in a higher cavity pressure, and with a higher cavity pressure, a more refined surface finish can be achieved on the formed part.

Analysis of the forged parts (as forged in accordance with the present invention and before any surface finishing or grinding or polishing) has shown that the surface finish or surface roughness of the forged parts is less than about 32 Ra. As known in the art, the Ra value is an amplitude parameter that characterizes the surface based on the vertical deviations of the roughness profile from the mean line (Ra is typically expressed in “millionths” of an inch, commonly referred to as “microinches”). The present invention thus provides a forging system that forges parts which, after forging and before any other surface finishing processes such as grinding or polishing, have a surface finish or surface roughness of less than 32 Ra, which provides a substantially smooth or finished part and results in a reduction or obviation of post-forging processing, including grinding and polishing of the forged part.

The forging system of the present invention also provides for enhanced material utilization, such as greater than 60 percent material utilization, preferably greater than 70 percent material utilization, and preferably greater than 80 percent material utilization (such as about 85 percent material utilization as achieved in sample testing), which is a significant improvement of the prior art forging methods, which typically result in less than 40 percent material utilization. Thus, the flash of the finished part as forged in accordance with the present invention, may be less than about 40 percent of the billet or forging stock material initially disposed in the die cavity at the onset of the forging process for an individual component, and preferably is less than about 20 percent of the billet or forging stock material initially disposed in the die cavity at the onset of the forging process for an individual component. Testing and computer modeling has shown that the forging process of the present invention may achieve about 80 to 85 percent material utilization, which is significantly greater than the 35 to 40 percent material utilization achieved via known split die forging processes. Such enhanced material utilization is a significant improvement in forging orthopedic implants out of biocompatible metals, particularly due to the high cost of such biocompatible metals, whereby an improvement in material utilization can substantially reduce the overall costs of the forged parts. For example, the forging system of the present invention may reduce the costs of the forged part by about 30 percent or more as compared to conventional forging systems.

The custom press or forging system or apparatus of the present invention, which is capable of supplying force along multiple axes, produces the enhanced or improved femoral knee component or orthopedic implant as discussed above. The press design provides sufficient and non-yielding clamp force to the form tooling inserts or die portions to assure that there is no flexing or give or separation of the die portions during the forging process so that the components can be made to the precise or proper specifications. Evaluation of various clamping techniques (mechanical, pneumatic and hydraulic) led to a hydraulic force device or actuator being preferred, due to a hydraulic actuator's ability to meet the unique high clamping characteristics of the forging process of the present invention. Also, the hydraulic clamp system of the forging system of the present invention operates to assure that the die inserts or portions are repeatably located in their precise or proper position relative to each other and relative to the form punch (which is controlled and moved by a vertical hydraulic piston or actuator that moves the punch in a direction generally transverse to the direction of movement of the die portions). To achieve the desired control of the die portions, the actuator or cylinder movement or operation and die portion position may be controlled with an integral delta control system and linear transducers, while the applied pressures or forces may be monitored by pressure transducers or the like.

Therefore, the present invention provides a multi-axis, high pressure precision forging system or apparatus. The high pressures achieved by the forging system during the forging process results in a forged component (forged out of a biocompatible metallic material) that has finer grain size and substantially homogeneous material composition with substantially uniform dispersion of the alloy elements throughout the forged component, which may lead to enhanced tribological interaction and enhanced wear and life cycles for the formed parts. The forged component thus may have a higher Rockwell hardness and a more homogeneous surface with reduced imperfections at or in the surface of the forged component. Thus, the high pressure forging system of the present invention may provide or forge or form an orthopedic implant component with a finished surface that that requires little or no machining to achieve the desired smoothness or surface quality. Such a finished grade surface quality to the forged part may provide reduced surface imperfections and may lead to a reduction or obviation of subsequent machining or polishing processes after the component is forged, and the component forged via the forging process of the present invention may only require machining along a peripheral edge of the product to remove the small amount of flash that is present on the forged element after it is removed from the die. The present invention thus provides a forging process that provides a component with reduced grain size, more uniform microstructure and enhanced surface qualities or characteristics, while substantially increasing material utilization and thus reducing the amount of scrap material or material wasted during the forging and thus reducing finishing processes and finishing operation hours of the orthopedic implant element.

By increasing the strength of the orthopedic insert, such as a femoral knee component, utilizing the improved forging process or system of the present invention, it is envisioned that the cross-section thickness of the forged component may be reduced, thereby providing for less bone removal of the lower end of the femur during surgery to implant the femoral knee component. Also, the more tightly compacted finer grain structure of a femoral component that is forged via the high pressure forging system/process of the present invention may provide enhanced or smoother surfaces (with reduced imperfections and the like at the surfaces) and thus may achieve enhanced wear qualities in the replaced knee, and may reduce deterioration of or wear at the articulating surface (typically a polyethylene component) of the element (at or part of the tibial plate) that is engaged by the articulating surface of the forged femoral component.

Changes and modifications in the specifically described embodiments may be carried out without departing from the principles of the present invention, which is intended to be limited only by the scope of the appended claims as interpreted according to the principles of patent law.

1. A method for forming an orthopedic implant element, said method comprising:
providing a forging die, said forging die comprising a punch and a split die having a first die portion and a second die portion, said first and second die portions comprising respective cavity portions, wherein said first die portion is movable relative to said second die portion between an opened state of said split die, where said first and second die portions are spaced apart to allow for removal of a formed orthopedic implant element from within said cavity portions, and a closed state, where said first and second die portions are secured together and said cavity portions cooperate to define a cavity to form said orthopedic implant element therein;
providing a billet comprising at least one of zirconium, a zirconium alloy, titanium, a titanium alloy, stainless steel, a stainless steel alloy, cobalt-chrome and a cobalt-chrome alloy;
inserting said billet into said cavity of said split die;
closing said split die via moving at least one of said first and second die portions towards and into engagement with the other of said first and second die portions;
moving said punch to deform said billet in said cavity to forge said orthopedic implant element in said cavity while said first and second die portions are substantially fixed relative to one another when said split die is in said closed state; and
opening said split die to remove said forged orthopedic implant element.

2. The method of claim 1, wherein moving said punch comprises moving said punch to generate a cavity pressure of at least 200,000 psi on said billet in said cavity to form said orthopedic implant element.

3. The method of claim 1, wherein moving said punch comprises moving said punch to generate a cavity pressure of at least 250,000 psi on said billet in said cavity to form said orthopedic implant element.

4. The method of claim 1, wherein closing said split die comprises actuating at least one hydraulic ram to move said first section relative to said second section, and wherein said at least one hydraulic ram is operable to maintain said split die in said closed state during said forging process.

5. The method of claim 4, wherein said at least one hydraulic ram comprises a first hydraulic ram for moving said first die portion and a second hydraulic ram for moving said second die portion.

6. The method of claim 5, wherein said first and second hydraulic rams maintain alignment of said first and second die portions as said first and second die portions are moved to close said split die such that said first and second cavity portions align and cooperate to establish said cavity when said split die is closed.

7. The method of claim 6, wherein said first and second hydraulic rams are moved along a die closing axis and wherein said punch is moved along a punch axis that is generally transverse to said die closing axis.

8. The method of claim 1, wherein moving said punch comprises moving said punch at least partially into said cavity with a first gap established between a first side of said punch and a portion of said first die portion adjacent said cavity and a second gap established between a second side of said punch and a portion of said second die portion adjacent said cavity, and wherein excess material of said billet exits said cavity through said first and second gaps as said punch is moved into said cavity to form said orthopedic implant element.

9. The method of claim 8, wherein gap dimensions of said first and second gaps are selected to establish a desired cavity pressure in said cavity when said punch is moved into said cavity to form said orthopedic implant element.

10. The method of claim 9, wherein said gap dimensions are reduced to increase said cavity pressure in said cavity during said forging process.

11. The method of claim 8, wherein gap dimensions of said first and second gaps are less than about 0.080 inches.

12. The method of claim 8, wherein gap dimensions of said first and second gaps are less than about 0.060 inches.

13. The method of claim 1, wherein said billet comprises at least one of zirconium and a zirconium alloy.

14. The method of claim 13, wherein said orthopedic implant element, when removed from said opened split die, has a grain size of about G16.0 or finer substantially throughout said orthopedic implant element.

15. The method of claim 1, wherein said orthopedic implant element, when removed from said opened split die, has a surface finish of no more than about 32 Ra.

16. The method of claim 1, wherein said orthopedic implant element, when removed from said opened split die, has flash material at and around the area at which the punch entered the cavity during the forging process.

17. The method of claim 16, wherein said orthopedic implant element has substantially no flash along its surfaces remote from said punch where the die portions mate together.

18. The method of claim 16, wherein said flash material comprises less than about 40 percent of said billet.

19. The method of claim 16, wherein said flash material comprises less than about 20 percent of said billet.

20. The method of claim 1, wherein said orthopedic implant element comprises a femoral knee component.

21. The method of claim 20, wherein said first cavity portion defines an anterior flange of said femoral knee component and said second cavity portion defines condyles of said femoral knee component and wherein each of said first and second cavity portions define a portion of an articulating lower surface of said femoral knee component.

22. The method of claim 21, wherein said orthopedic implant element, when forged and before finishing, has flash material partially at an upper end of said anterior flange and at an upper end of said condyles.

23. A method for forming an orthopedic implant element, said method comprising:
providing a forging die, said forging die comprising a punch and a split die having a first die portion and a second die portion, said first and second die portions comprising respective cavity portions, wherein said first die portion is movable relative to said second die portion between an opened state of said split die, where said first and second die portions are spaced apart to allow for removal of a formed orthopedic implant element from within said cavity portions, and a closed state, where said first and second die portions are secured together and said cavity portions cooperate to define a cavity to form said orthopedic implant element therein;
providing a billet;
inserting said billet into said cavity of said split die;
closing said split die, wherein closing said split die comprises actuating at least one hydraulic ram to move said first section relative to said second section, and wherein said at least one hydraulic ram is operable to maintain said split die in said closed state during said forging process;
moving said punch to deform said billet in said cavity to forge said orthopedic implant element in said cavity while said first and second die portions are retained together via said at least one hydraulic ram when said split die is in said closed state;
wherein moving said punch comprises moving said punch to generate a cavity pressure of at least 200,000 psi on said billet in said cavity to form said orthopedic implant element; and
opening said split die to remove said forged orthopedic implant element.

24. The method of claim 23, wherein said billet comprises at least one of zirconium, a zirconium alloy, titanium, a titanium alloy, stainless steel, a stainless steel alloy, cobalt-chrome and a cobalt-chrome alloy.

25. The method of claim 23, wherein moving said punch comprises moving said punch at least partially into said cavity with a first gap established between a first side of said punch and said first die portion of said split die and a second gap established between a second side of said punch and said second die portion of said split die, and wherein excess material of said billet exits said cavity through said first and second gaps as said punch is moved into said cavity to form said orthopedic implant element.

26. The method of claim 25, further comprising selecting a first gap dimension of said first gap and a second gap dimension of said second gap to establish a selected cavity pressure during forming of said orthopedic implant element.

27. The method of claim 26, wherein said first and second gap dimensions are selected to be less than about 0.080 inches.

28. The method of claim 26, wherein said first and second gap dimensions are selected to be less than about 0.060 inches.

29. The method of claim 25, wherein said excess material comprises less than about 40 percent of said billet.

30. The method of claim 25, wherein said excess material comprises less than about 20 percent of said billet.

31. The method of claim 23, wherein said orthopedic implant element, when removed from said opened split die, has a grain size of about G16.0 or finer substantially throughout said orthopedic implant element.

32. The method of claim 23, wherein said orthopedic implant element, when removed from said opened split die, has a surface finish of no more than about 32 Ra.

33. A forged orthopedic implant element comprising:
a material selected from the group consisting of zirconium, a zirconium alloy, titanium, a titanium alloy, stainless steel, a stainless steel alloy, cobalt-chrome and a cobalt-chrome alloy; and
a grain size of about G16.0 or finer substantially throughout said orthopedic implant element.

34. The orthopedic implant element of claim 33, wherein said orthopedic implant element comprises a femoral knee component, and wherein said femoral knee component comprises an anterior flange and condyles and an articulating lower surface.

35. The orthopedic implant element of claim 33, wherein said material comprises at least one of zirconium and a zirconium alloy.
